(19) United States
(12) Patent Application Publication
(10) Pub. No.: US 2007/0197906 A1
(43) Pub. Date: Aug. 23, 2007

(54) Magnetic field shape-adjustable medical device and method of using the same

(76) Inventor: Rogers C. Ritter, Charlottesville, VA (US)

Correspondence Address: HARNESS, DICKEY, & PIERCE, P.L.C, 7700 BONHOMME, STE 400, ST. LOUIS, MO, 63105, US

(21) Appl. No.: 11/653,778

(22) Filed: Jan. 16, 2007

Related U.S. Application Data
(60) Provisional application No. 60/761,499, filed on Jan. 24, 2006.

(51) Int. Cl. A61B 5/05

(52) U.S. Cl. 600/424

(57) ABSTRACT

A medical catheter or guide wire comprising one or a plurality of combinations of magnetically permanent or permeable elements attached to an elongated structural element, the medical device being controllably bendable at the element combination(s) upon application of a magnetic field generally in the direction of the local element long axis.

### CROSS-REFERENCE TO RELATED APPLICATIONS

This application claims the benefit of U.S. Provisional Patent Application Ser. No. 60/761,499, filed Jan. 24, 2006, the disclosure and claims of which are incorporated herein by reference.

### FIELD

This invention relates to the navigation of guide wires, catheters, and similar interventional medical devices, and in particular to a system for and a method of adjusting the shape of such devices, including the generation of tight device bends, by application of an external magnetic field.

### BACKGROUND

Interventional medicine is the collection of medical procedures in which access to the site of treatment is made by navigation through one of the subject's blood vessels, body cavities or lumens. Interventional medicine technologies have been applied to manipulation of medical instruments such as guide wires and catheters which contact tissues during surgical navigation procedures, making these procedures more precise, repeatable and less dependent on the device manipulation skills of the physician. Some presently available interventional medical systems for directing the distal tip of a medical device use externally generated magnetic fields. In many interventional medicine applications, and particularly with small devices such as guide wires, it is desirable for the device to make tight turns, such as when following the convoluted path of a small artery or when attempting to make a turn at a vessel branch.

In the “standard” mode of navigation with one magnet at the tip of a catheter or guide wire (generically call this “catheter”) the shape of the bent catheter either in a hollow vessel, e.g. the heart, or in a segment of a vessel with a bit of free space for the catheter, the bend is a cooperation of a variety of torques. These include the magnetic torque at the tip, the mechanical restraining torque of any free segment of a catheter, and the mechanical torque applied by a vessel wall, or a segment of a wall. These can lead to an overall shape of the catheter which might promote excessive friction at a region of a vessel wall, or they might promote prolapse of the catheter. If a catheter can be made to curve intrinsically upon application of an external magnetic field and in varying degrees depending on the component of the field along the catheter axis at a point, these tendencies may be overcome.

In several cases it would be useful to be able to bend a stent delivery wire or other interventional device in order to reduce friction on a vessel wall on the outside of the bend. FIG. 1 illustrates such a situation and the use of prior-art devices in magnetic navigation; similar situations occur frequently in conventional (mechanical) navigation as well. In the essentially uniform fields provided by magnetic navigation systems within an operating region at a given time, the requirement for the device tip to re-orient and bend markedly to make tight turns has led to the use of flexible Platinum Cobalt (PtCo) coil permanent magnets or the use of multiple Neon Iron Boron (NeFeB) magnets. PtCo suffers from relatively low magnetization, and NeFeB cannot be made flexible and yet retain significant magnetization strength. In principle, the use of a series of multiple magnets as illustrated in FIG. 1 could provide effective bending over an extended guide wire length since the wire can be bent between the magnets. However, when immersed in a magnetic field, each individual magnet tends to re-orient such that its magnetic moment is aligned with the local direction of the magnetic field; accordingly, a series of multiple magnets placed in a uniform field tends to stiffen towards alignment. This tendency limits the bending effectiveness of such of a device in use in a magnetic navigation system. In practice, and upon application of a magnetic field initially at a large lead angle to the device distal end, the most distal magnet elements re-orient a certain amount against the wire bending torque resistance, and upon mechanically settling the next most distal element is then immersed in a field at a smaller lead angle than the leading element originally was, thus being subjected to a reduced torque; and so on down the set of magnetic elements. Accordingly it would be desirable to design a device that can be bent at a known radius of curvature over a pre-specified distance upon application of a magnetic field.

### SUMMARY

In general, there is not a known magnetic device which will bend internally upon the application of a uniform magnetic field. In particular, there is no known navigation device that will bend upon application of a magnetically field in a direction generally parallel with the device long axis. The present invention discloses such devices and methods of using the devices in navigation. It describes a device inserted along a catheter or guide wire that will bend in response to the application of an externally generated magnetic field. In circumstances where permitted, it may be used in conjunction with magnetically navigated catheters having one or more magnets at or near the tip of the catheter. In such cases the navigating magnetic field will be the same externally applied bending field, and the device must be capable of being twisted about its axis, most likely from the proximal end by the physician. If the device orientation cannot be determined by the imaging system used in the navigation, some means of marking it must be employed. Additionally, the devices of the present invention can be used as navigating elements at the tip of a catheter or guide wire.

Magnetic elements are applied at appropriate locations as a segment of a guide wire or catheter, each magnetic element having desired length or tapered bending properties. The device magnetic elements can consist of a number of different means of locally applying a magnetic field gradient to an adjacent permeable magnetic disc, which when its magnetic moment is made large by application of an external magnetic field, is attracted to the adjacent source of the gradient, thereby bending a “spine” attached to the edges of the elements. It can be comprised for example of different axial arrangements of preferably thin cylindrical discs, which may be made of permeable or permanent magnetic materials. Preferably the array would be made of appropriate mixtures of discs of these two types of materials. In another embodiment the array can consist of a spine along the edge of a coil of permeable magnetic wire.

The magnetic element or elements can be used with catheters and guide wires that are not magnetically guided, and with some limitations, with such devices that have guiding magnets at or near the tip region. For example and as schematically illustrated in FIG. 2, the element could be used near the tip of a guide wire so that it could be navigated rapidly and with simple magnetic field application by the physician, the magnetic field being applied only when needed for a tight turn. In another application, as in FIG. 3, when a known region of difficult curvature in a vessel causes friction with a typical guide wire, the application of a field can bend the guide wire away from the problem wall, when a tip magnet cannot.

According to the present invention devices are described that use torques exerted on adjacent discs of a magnetic element, held separated by an edge-attached resilient element (e.g., a wire), to create a bending of the edge element by attraction between the discs. The resilient element can be considered the “spine” of the magnetic element. Generally, a magnetic moment m immersed in a uniform magnetic field B will tend to align with the magnetic field: the magnetic field exerts a torque \vec{m}×\vec{B} that tends to re-orient the magnetic moment towards alignment with the field. However a magnetic moment, or a magnetic dipole, immersed in a varying magnetic field will be subjected to forces that are a function of the magnetic field gradient. If m denotes the moment of a dipole or of a permeable disc, the force Fₓ along axis x (for example chosen to coincide with the local device long axis) is given by Fₓ=m.grad(Bₓ) (and similar expressions for forces F_{y} and Fₓ along axes y, z chosen to define an orthonormal vector set). The resulting force is strongest where the field magnitude varies the fastest and where the magnetic moment is aligned with the direction of greatest field variation. A magnetized disc attached on its edge to the spine or wire and subjected to a varying field will be subjected to forces that will effect a torque for the disc with respect to its point of attachment on the spine; as a result, a bending of the edge elements is generated by attraction or repulsion of the discs. One advantage of the present invention is that it provides a way for a magnetic element to bend along its length upon the application of a uniform external magnetic field, in particular upon application of a magnetic field generally parallel to the local device long axis, and uses the proximity of the elements or discs to increase the sensitivity of the unit to external fields.

### BRIEF DESCRIPTION OF THE DRAWINGS

FIG. 1 is a schematic diagram showing magnetic navigation of a guide wire according to the prior art;

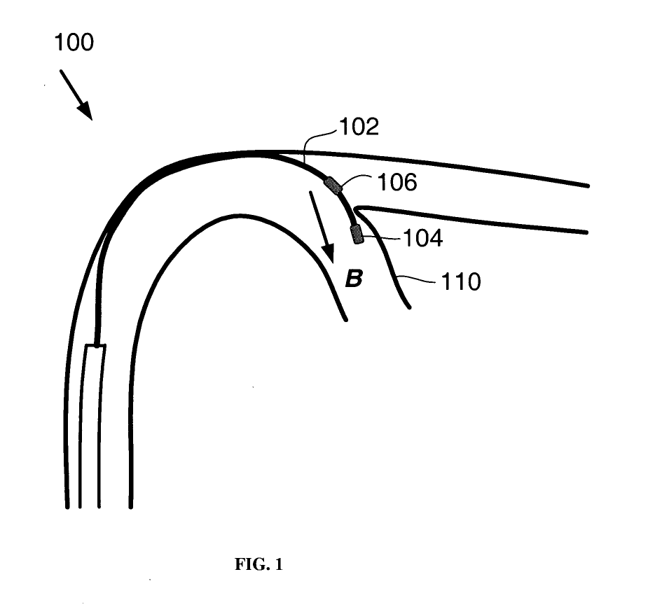

FIG. 2 is a schematic diagram showing an embodiment of the present invention and application to bending the distal end of a guide wire;

FIG. 3 is a schematic diagram showing another embodiment of the present invention to reduce device friction along a vessel wall;

FIG. 4 is an enlarged side elevation view of a device constructed according to the principles of the present invention, using permeable discs;

FIG. 5 presents magnetization curves B-H for a number of materials;

FIG. 6 is an enlarged side elevation view of a device according to the principles of the present invention, using a magnetic coil;

FIG. 7 is an enlarged side elevation view of a device constructed according to the principles of the present invention, using alternating permanent and permeable discs; and

FIG. 8 presents the fluxmetric demagnetization factor N_{f} and the magnetometric demagnetization factor Nₘ along the axis of a cylinder as a function of the length-to-diameter ratio.

Corresponding reference numerals indicate corresponding parts throughout the several views of the drawings.

### DETAILED DESCRIPTION

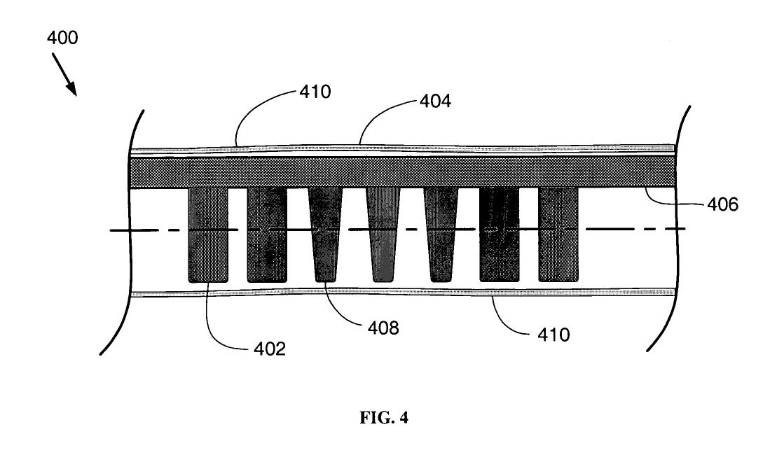

In one embodiment of the present invention, multiple magnetic elements are attached edge-wise onto a flexible member. One such element generally indicated by numeral 400 consists of a series of permeable magnet discs 402 attached by the flexible member, such as a wire 404, along one edge 406, as shown schematically in FIG. 4. The flexible edge wire is designed to exhibit only small bending in response to typical mechanical torques, but the application of an externally induced magnetic field in excess of a predetermined design value, will provide strong inter-disc attraction so that the edge wire “spine wire” bends. Magnetic navigation systems available from Stereotaxis, Inc., utilizing either electromagnets or permanent magnets are capable of projecting magnetic fields in the operating region in any direction. Such systems are capable of projecting field strengths of at least 0.06 T, and in some cases 0.08 and in some cases 0.1 T in the operating region in the subject. In this embodiment the device is preferably designed to respond to a field of 0.08 T. The discs are permeable magnet discs with appropriate B-H curves so that application of an external field will result in the generation or increase of their magnetic moments and consequently axial attraction between them. The close proximity of the discs means that significant forces can be attained between them with relatively small magnitude fields, such as the 0.08 Tesla field presently provide by the Niobe® magnet system, available from Stereotaxis, Inc., St. Louis, Mo.

The attracting gradients acting between neighboring pairs of permeable discs can be thought of as the gradient attraction between two dipoles. This interaction will have a force inversely proportional to a high power of the distance between centers of the two discs, the power depending on geometry, i.e., size and spacing. In this embodiment, the force between neighboring permeable discs can only be attractive, and in essence will assume a quadratic behavior. A useful feature of some aspects of this invention is that the spacing necessary for tight bends can still be such that a large amount of navigating magnetic moment is available for ordinary navigating turns as well. If the bend of the element would be constrained by the spacing, the faces of the discs can be shaped (e.g., tapered) for even tighter bending as shown for disc 408. The choice of permeable magnetic material will influence the device operational properties: the permeability, combined with the spacing and size of discs, will determine the amount of bending for an applied field of given magnitude and direction.

FIG. 4 shows a row of such discs schematically, with the attaching spine at the top. The discs are shown on edge in the Figure, and for illustration a nominal spacing one-half that of the disc thicknesses is used. It is apparent that the diameter and length, as well as disc thickness and spacing, can be made different for a variety of requirements. It is also apparent that spacing and disc thicknesses can be varied if desired in a given application, so that a bend can be accomplished with a variable radius of curvature along the length of the device. The stiffness of the “spine wire” is designed to provide the appropriate degree of bend for the applied magnetic field. For several reasons, it may be desirable to provide a sleeve of flexible material 410 around the magnet array. This can be useful in preventing any sharp edges of the discs from catching and/or damaging the delicate blood vessel wall. Such a sleeve will further tend to keep the discs in line so that the spine does not twist significantly, but will only bend.

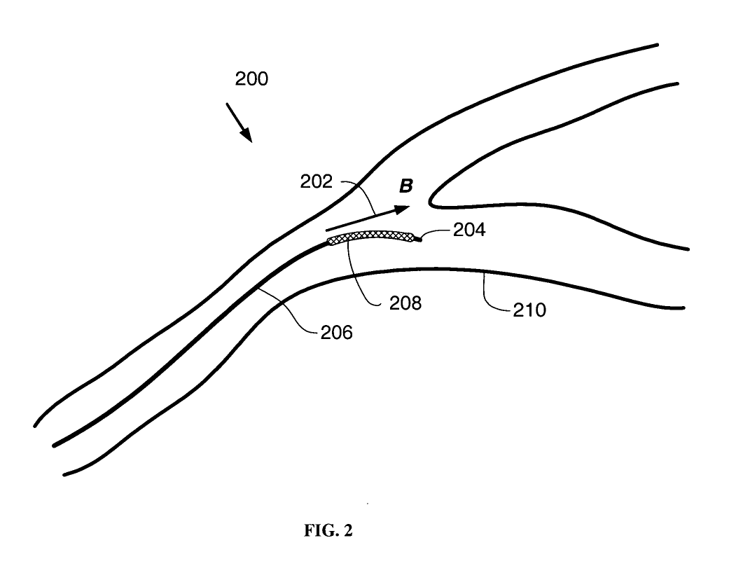

FIG. 2 illustrates how application of an externally generated magnetic field B, 202, bends the tip 204 of a guide wire 206 designed according to the principles of the present invention. The magnetic element 208 schematically shown by cross hatches bends the guide wire to orient the tip 204 in the desired vessel branch 210.

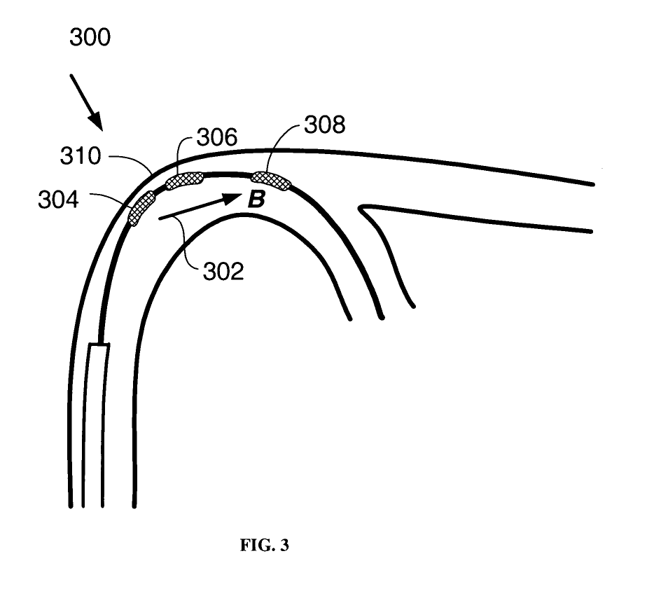

Similarly, FIG. 3 illustrates how application of a magnetic field 302 to a number of schematically represented magnetic elements 304-308 designed according to the principles of the present invention can bend a section of a guide wire and reduce friction of the device along vessel wall 310.

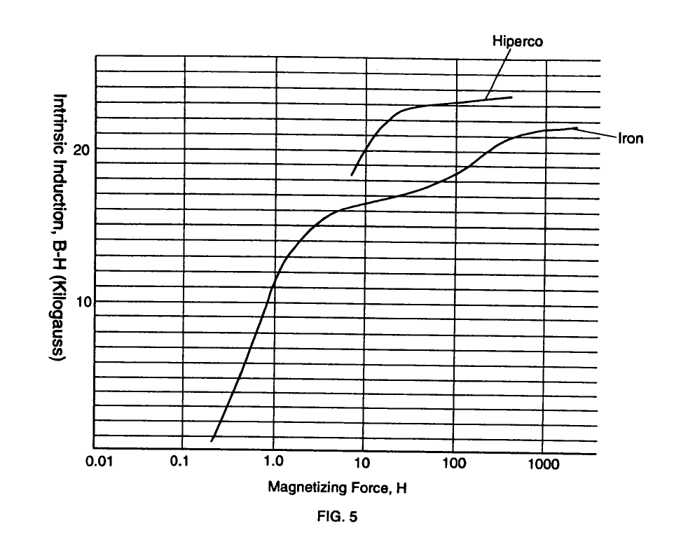

FIG. 5 is a plot of the magnetizing curves, B vs. H, of a number of permeable materials. It is seen that a magnetizing force H of 800 oersteds (about 64,000 A/m in SI units) crosses material 20 (grey cast iron) at a point where the slope (its permeability) is about 5 μ₀ (in SI units). A variety of other materials, not shown on this chart, can be found to have significant permeability at this level of field. It is to be noted that the “free field” application of such data to this element actually requires a complex calculation involving the reluctance associated with this particular geometry. The reluctance characterizes the opposition offered in a magnetic circuit to magnetic flux and is proportional to the element length and inversely proportional to the product of the element cross sectional area and the material permeability; thus, highly permeable materials exhibit a reduced reluctance. Such a “free-field” application calculation can be made by finite element methods.

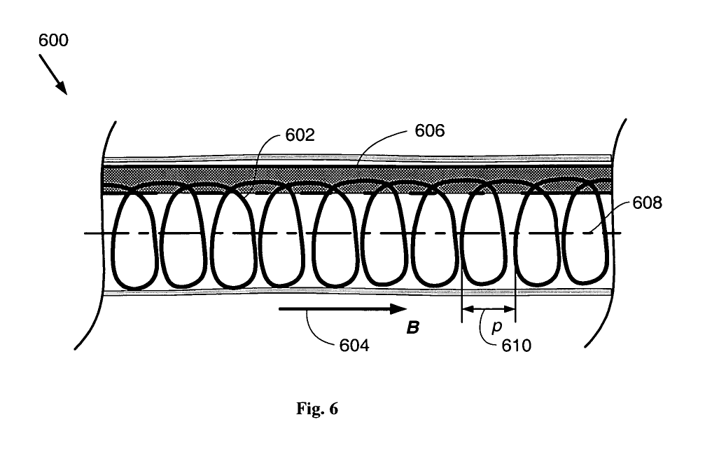

Another, simpler, embodiment of the bendable magnetic element uses a coil of magnetic wire instead of the discs, as illustrated in FIG. 6. This coiled wire 602 can be made of a permeable magnetic material. Application of an external magnetic field 604 with a component along the axis of the coil will result in a tendency of the coil to contract in length, each turn attracting the next. The coil comprises a spine 606 attached to each turn along one side, parallel to the axis 608 of the coil, so that the contracting effect results in a bend of the spine. The pitch of the coil 610 is designed to optimize the amount of magnetic material per unit length, while at the same time leaving enough space to permit bending on the inside of the curve (opposite the side of the spine).

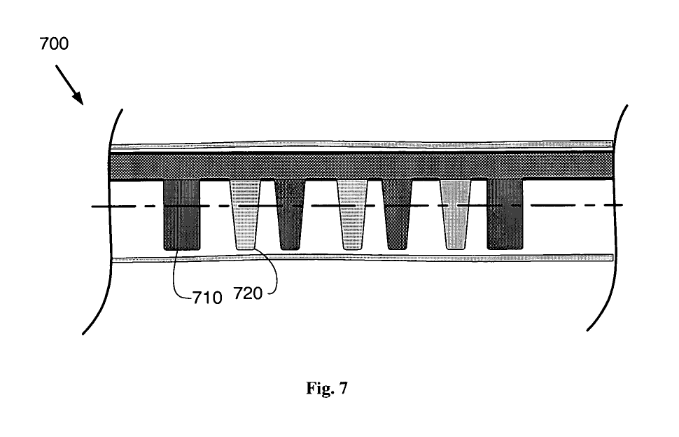

In a third embodiment illustrated as 700 in FIG. 7, the discs are made alternately permanently magnetic and permeable material. This arrangement results in a much stronger element bending capability, as the moment of a permanent disc magnet can attain 1.2 T. In comparison, the dipole-to-dipole attraction of the implementation of FIG. 4 is weaker because an externally applied field of magnitude of the order of 0.08 T cannot induce such a high permeable disk moment.

Moreover, such an arrangement can permit some design and operation independence not possible with the totally permeable disc embodiment. It can be seen that the application of an element-bending external field might at times interfere with a navigating field. Therefore it would be advantageous to have a variety of different geometries of this bendable element. For example, permanent magnets could in some cases be of alternate or occasionally alternate magnetization directions, so that some would exhibit repulsion and some attraction. Spacing variation, thickness variation and other variations could permit special purpose versions of bendable elements to cope with bending and guiding interference cases.

An arrangement of one preferred embodiment consisting of alternating permanent and permeable discs of a bendable magnetic element can have several optional features. It can, depending on the design and the size of the external magnetic field, enable field-direction dependent bending of the element. For an “opposing” direction of the magnetic field (relative to the direction of the field in the permanent magnet elements, and if strong enough) the magnetization of the permeable discs would repel the adjacent permanent magnet discs rather than attract them. For an “aiding” field direction all discs would attract, thus bending the spine in the opposite direction. In the repulsion mode the permanent magnet discs could be designed to magnetize the permeable discs only slightly in one direction, and the application of an external magnetic field would magnetize the permeable discs in the opposite direction from that of the permanent discs, thus causing the repulsion. Thus the bend of the bendable element spine can be made to occur in one direction or its opposite just by changing the direction of the externally applied field. By choosing field angles the total angle of these bends can be controlled. Biasing of the element could be chosen so that it was pre-bent in one direction, which could be removed or reversed by application of the external magnetic field. Such an application might be particularly useful at the device distal end. In such an application, it might alleviate the need to mechanically rotate the guide wire with respect to its long axis, as required in conventional navigation to orient the bent tip in a desired direction. In other embodiments the thickness, spacing or other element parameters can be adjusted in consideration of both the external field to be applied and of the material chosen for the permeable discs, as well as of the sharpness of bend required for a given applied field. The number of elements and their extension along the spine will determine the total bend of the unit. A feature of all embodiments is that the amount of bending can be controlled by the angle of the externally applied field relative to the axis of the discs or coil.

A first exemplary embodiment of this preferred approach for a bendable element is illustrated in FIG. 7 and consists of alternate discs being 710 of a permanent magnetic material such as NdFeB (Neodymium Iron Boron), and permeable discs 720 of an element such as Hiperco. These discs are not regularly spaced in order to maximize the bending capability of the element for external fields weaker than the fields at a given disc spacing of the permanent magnet elements. If the spacings were uniform, each permeable disc would fall between two permanent magnet discs that would act somewhat like a Helmholtz coil arrangement. Such an arrangement provides a relatively uniform field at the center, which would be applied to the permeable disk. This disc would experience a uniform, or nearly uniform, field and it then would not be attracted either way, or at most be weakly attracted one way if the spacing were not exact. In current magnetic navigation systems the externally applied field in general is very uniform in magnitude and locally in direction and therefore cannot be depended upon to generate a local field gradient. In the example below, it will be shown how to avoid this situation by appropriately changing the spacing of the discs each side of the permeable discs. It can be seen that similar results could be obtained by alternating the thickness (and therefore strength) of the permanent magnet discs. In either case, detailed optimization can maximize the efficiency of bending, that is, the amount of bending per unit of the repeating pattern for a given applied external field.

In designing this bending system it is necessary to know within reasonable accuracy the size of permeable disc in order to know the change in its moment induced by an external field, which in turn causes it to react to the locally generated gradient from the adjacent permanent magnet discs. The aspect ratio (the thickness, i.e., length-to diameter ratio) of the permeable material will determine the magnetic moment and the response to the gradient. It is well known that this aspect ratio strongly affects the “demagnetizing factor” N of the disc. One method of determining the moment of this disc as a function of the total field present it to use a reference response of a permeable cylinder of the same magnetic material to an applied field in conjunction with well known theory for relating the demagnetizing factor of the disc to that cylinder. In using this method, it has been found by measurement that a reference permeable cylinder of Hiperco 6 mm long and 2.5 mm diameter will develop a magnetic moment of about 0.56 A-m² per Tesla of applied field in an open field arrangement, for applied fields up to about 0.3 Tesla, i.e., near saturation. Another cylinder this size of 0.9999 pure iron exhibited a slope of about 0.22 A-m² per Tesla up to about 0.3 Tesla in this open field arrangement. Still another cylinder of annealed pure soft iron of this size exhibited a slope of about 0.35 A-m² per Tesla at fields up to about 0.3 Tesla. This information can be used to estimate the performance of a practical device. In the case described below Hiperco is used as the reference cylinder.

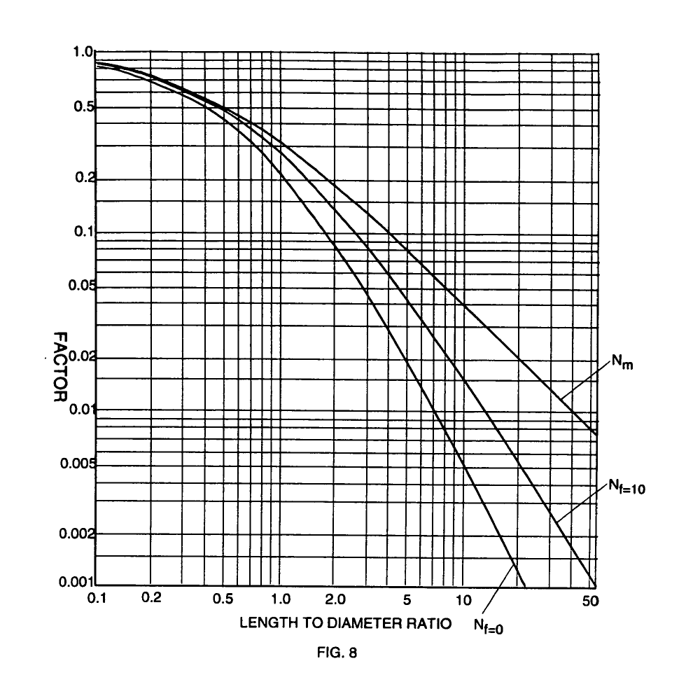

It is known in the field that the dependence of the demagnetizing factor N on the aspect ratio of a permeable ellipsoid (and of a cylinder) can be accurately calculated. For an ellipsoid the internal field is uniform, and the factor is exact. For a cylinder or disc the field is not everywhere uniform, and approximations are necessary in the calculation—in particular the axial and transverse factors differ. Thus the demagnetizing factor affects the moment and the attractive or repulsive force of an applied field gradient (generated by the permanent discs) acting on the permeable disc in question as well as the response of the permeable disc to the externally applied field. FIG. 8 shows the geometrical effects, that is, the calculated demagnetizing factors for permeable cylinders assumed to have material that has linear response to the applied field. This is also illustrated in Experimental Methods in Magnetism, H. Zijlstra, North-Holland Publishing Company, Amsterdam, 1967, page 70, incorporated herein by rerence. Values of the abscissa below unity refer to discs, which have a higher demagnetizing factor than long cylinders. In FIG. 8 the flux-metric calculations, N_{f}, show the appropriate demagnetizing effect for the cylinder and disc magnetized along the axis. (The symbol N is used for this factor in the following as in the paragraphs above.) These curves assume a uniform susceptibility [symbol] for the material, which is approximately but reasonably the case for the fields and materials in this invention. We shall use that assumption in the following, and in addition assume that [symbol] is independent of the applied field. This assumption has been shown by experimental measurements in Hiperco showing that up to approximately 2 Tesla the magnetic moment of the elements increases linearly.

The cylinder magnetization M (assumed to be directed along the axis) can be reduced by the demagnetization according to:
M=K(H₀+H_{d}), (1.1)
where H₀ is the applied (assumed homogenous) external field and H_{d} is the (negative) demagnetizing field. The demagnetizing factor N is defined by
μ₀H₀=NM (1.2)
From these and the above assumptions the dependence of M on susceptibility [symbol] is given (Zijlstra) by:
M=μ₀ [symbol] _{z}H₀/(1+μ₀ [symbol] _{z}N), (1.3)
where [symbol] _{z} is the susceptibility along the axis and is equal (μ/μ₀−1) and where μ/μ₀ is the relative permeability of the material. The magnetic moment m_{c} of a measured reference cylinder will be approximately the magnetization M_{c} times the volume V_{c}, and that for the disc m_{d}=M_{d}V_{d}. Thus the ratio of disc to cylinder moments for a given material will have the numerators of the above equation canceling, so that
m_{d}/m_{c}=(v_{d}/V_{c})×((1+μ₀ [symbol] _{z}N_{c})/(1+μ₀ [symbol] _{z}N_{d})). (1.4)
The permeable discs used will have a permeability (and product μ₀ [symbol] _{z}) much greater than unity. By measurement this product is about 120. Thus the ratio of moments for a given applied field is approximately
m_{d}/m_{c}=V_{d}N_{c}/V_{c}N_{d}.
That is, the ratio of moments is approximately proportional to the volumes and inversely proportional to the demagnetization factors. Further assuming linearity for both these cases, which is well established in the magnetic fields considered here, and given a zero magnetization in the absence of applied field, the slopes of moments versus applied field will obey the same relations:
S_{d}/S_{c}=V_{d}N_{c}/V_{c}N_{d}.

From this reasoning, the experimental data for Hiperco, above, can be geometrically ratioed for the permeable discs in this embodiment. In this example discs will be b 0.014″ diameter, the alternating permanent magnet discs 0.007″ thick and the permeable magnet discs 0.007″ thick. The spacings in this example will be 0.004″ on one side of the permeable disc and 0.006″ on the other. For simplicity the permanent discs will be chosen to be magnetized in the same direction in this example. Considering one segment of two permanent discs and the permeable disc between them, the repetition rate for each pattern is the 0.007″ permeable disc thickness plus 0.004″ and 0.006″ spacings plus one half the thickness of each permanent magnet, 0.0035″+0.0035″, adding up to 0.017″, which will be called Δx. The choice of design details of this geometry to optimize bending efficiency is complex, so the following is only an approximation for illustration.

Considering one segment of the three discs, the closest permanent magnet disc 0.014″ diameter by 0.007″ thick will project an axial field of about 0.118 T at a point on the axis about 0.0075″ distant from its face, which would be the center of the permeable disc. The further permanent disc will project about 0.08 T at the same plane. Thus a total of 0.198 T is applied by both permanent magnets. A constant gradient is acting on the permeable disc because of the different distances of this disc from the two adjacent permanent magnet discs. The gradient in this example is about 350 T/m. Application of a uniform external magnetic field, say of 0.08 Tesla, will not change the gradient, but will increase the moment in the permeable disc, as will be shown numerically in the following.

First, the calculation of the moment of the permeable disc will be shown. From FIG. 8 it is seen that the demagnetizing factor N is about 0.15 for the 6 mm by 2.5 mm cylinders described above, while it is about 0.7 for a disc having a thickness of one-half its diameter. It can be expected that a permeable disc 0.007″ thick will then have a demagnetizing factor of 0.7, or about 4.5 times that of the 6 mm cylinder. Using this along with the fact that the magnetic moment is essentially proportional to the volume, the expected moment of the disc can be calculated by comparison with the measurements given above for cylinders. From the dimensions of the example here, the referenced Hiperco 6 mm long cylinder has a volume about 1650 times that of the 0.007″ (0.178 mm) thick permeable disc. Using this smaller disc volume, together with the higher shape-dependent 4.5 factor of demagnetization in the above equation, the disc has an estimated moment in a given field is about 7,500 times smaller than this cylinder. Thus the factor 0.56 A-m² per Tesla for the reference cylinder is divided by 7500, to yield 7.5×10⁻⁵ A-m² per Tesla for the Hiperco disc in the bending element. A magnetizing field of about 0.198 T from the two permanent magnet discs plus 0.08 T externally applied will result in a moment of 2.1×10⁻⁵ A-m² in the permeable disc. From standard bending of a circular wire, the angle of bend of one section consisting of the disc triplet can be calculated from the equation:
Δθ=Δτ×(Δx/EI)radians. (1.5)
Here Δx is the element length, 0.017″ in our example, Δα is the torque created by the bending field in one element length, E is the Young's modulus of the spine rod and I its moment of inertia. For a simple, rough calculation, the rod is assumed to be circular, so I˜πr⁴/4. For typical stainless steels, E˜1.6×10¹¹ N/m².

In an example a bendable element 2 cm long is desired to bend about 30 degrees, roughly ½ radian. Each element length Δx is 0.043 cm, so approximately 46 such increments are needed. Assuming that each bends equally (only approximately true near the ends), the increment Δθ=0.5/46=0.0109 radians. Assuming that the external field is along the axis of the element, the bending torque Δτ is the force on the permeable disc times ½ the disc diameter. This is exerted over ½ of the segment length, so an effective length Δx′ will be 0.5×Δx=0.0085″. Inserting the above values in the equation for calculating method, however we find that the spine radius r˜0.8×10⁻³ or 0.0008″. A wire of 0.0008″ diameter would be rather small for azimuthal stability, and a preferable rod element would have a rectangular cross section. The aspect ratio of the cross section could easily be adjusted to provide softer bending but stiffer azimuth twisting.

The advantages of the above described embodiments and improvements should be readily apparent to one skilled in the art, as to enabling the controllable bending of a medical device such as a catheter or guide wire upon application of a magnetic field. Additional design considerations may be incorporated without departing from the spirit and scope of the invention. Accordingly, it is not intended that the invention be limited by the particular embodiment or form described above, but by the appended claims.

What is claimed:

1. A system comprising one or a plurality of combinations of magnetically permanent or permeable elements attached to an elongated structural element, said combinations providing means for bending the elongated structural element upon application of a magnetic field.

2. The system of claim 1, wherein the magnetically permanent or permeable elements are cylinders.

3. The system of claim 2, wherein the cylinders are discs.

4. The system of claim 1, wherein the magnetically permanent or permeable elements are attached on edge to the structural element.

5. The system of claim 1, wherein the structural element comprises one of the set consisting of a rod, a wire, a tube.

6. The system of claim 5, wherein the design of the structural element provides a means for the control of the amount of torsion along the structural element long axis.

7. The system of claim 1, wherein in each combination of magnetically permanent or permeable elements the parameters of the combination, including shape, separation, material composition, permanent element orientation, are selected to provide means for bending the structural element at a specific radius of curvature when subjected to a magnetic field of known magnitude and direction.

8. The system of claim 7, wherein the shapes of the magnetically permanent or permeable elements are tapered to provide means for increased bending of the structural element.

9. The system of claim 3, wherein the discs are alternating permanent and permeable discs spaced to provide means for bending the structural element at a specific radius of curvature when subjected to a magnetic field of known magnitude and direction.

10. The system of claim 9, wherein the alternating combination of permanent and permeable discs provides a means for bending the structural element in one direction upon application of a magnetic field in one direction along the local element axis, and for bending the structural element in the opposite direction upon reversing the direction of the magnetic field.

11. The system of claim 1, providing means for selectable bending of the structural element upon variation of the applied magnetic field direction or magnitude.

12. The system of claim 1, providing means for bending of the structural element upon application of a magnetic field generally in the direction of the local structural element long axis.

13. The system of claim 3, wherein the discs are made of a permeable material.

14. The system of claim 5, wherein the rod has a circular cross-section.

15. The system of claim 5, wherein the rod has a rectangular cross-section.

16. The system of claim 1, wherein one or a plurality of the magnetically permanent or permeable elements comprises a magnetic coil.

17. The system of claim 1, further comprising a sleeve placed around the magnetically permanent or permeable elements.

18. A method for bending a medical device comprising one or a plurality of combinations of magnetically permanent or permeable elements attached to a structural element, comprising:
(a) selecting a combination of magnetically permanent or permeable elements; and
(b) applying an externally generated magnetic field of known magnitude and orientation to the combination of magnetically permanent or permeable elements selected in (a).

19. The method of claim 18, wherein the magnitude and orientation of the externally applied magnetic field are chosen based on knowledge of the position and orientation of the selected element long axis.

20. The method of claim 18, wherein the amount of bending is controlled by the magnitude and orientation of the externally applied magnetic field

21. The method of claim 18, wherein the direction of bending is controlled by the magnitude and orientation of the externally applied magnetic field.

22. The method of claim 18, wherein bending is achieved upon application of a magnetic field generally in the direction of the local element long axis.

23. The method of claim 18, wherein application of a magnetic field enable orientation of a catheter or guide wire tip.

24. The method of claim 18, wherein application of a magnetic field enables reduction of the friction of the medical device with a vessel wall.

25. A method of designing a medical device comprising one or a plurality of combinations of magnetically permanent or permeable elements attached to a structural element, bendable upon application of a magnetic field, comprising:
(a) calculating parameters of the magnetically permanent or permeable elements, including shape, orientation, separation, and material composition;
(b) calculating parameters of the structural element, including shape, material composition, and dimensions
whereby a given amount of medical device bending is achieved upon application of a magnetic field of known magnitude and direction.

26. A elongate medical device having a distal end shapable by the application of a magnetic field to facilitate navigation of the distal end of the device in an operating region in a subject, the medical device comprising:
at least one section comprising a plurality of magnetically responsive elements at the distal end of the device, the elements secured together along one side in a longitudinally spaced relationship by a flexible element, the elements being magnetically responsive to attract each other upon the application of a magnetic field to assume longitudinally curved configuration, curving opposite the flexible element.

27. The medical device according to claim 26 wherein the magnetically responsive elements comprise a magnetically permeable material.

28. The medical device according to claim 26 wherein the magnetically responsive elements alternately comprise a magnetically permeable material and a permanent magnetic material.

29. The medical device according to claim 26 wherein the elements have a greater longitudinal dimension adjacent the flexible element, and oppose from the flexible element.

30. The medical device according to claim 26 wherein the magnetically responsive elements are similarly shaped.

31. The medical device according to claim 26 wherein the magnetically responsive elements are equally spaced.

32. The medical device according to claim 25 wherein the magnetically responsive elements are not equally spaced.
